(19) United States
(12) Patent Application Publication
(10) Pub. No.: US 2018/0221315 A1
(43) Pub. Date: Aug. 9, 2018

(54) COMBINATION THERAPY USING ACAMPROSATE AND D-CYCLOSERINE

(71) Applicant: Confluence Pharmaceuticals, LLC, Carmel, IN (US)

(72) Inventors: Steven L. JOHNS, Carmel, IN (US); Craig A. ERICKSON, Wyoming, OH (US)

(21) Appl. No.: 15/749,705

(22) PCT Filed: Aug. 4, 2016
(86) PCT No.: PCT/US2016/045547

§ 371 Date: Feb. 1, 2018

Related U.S. Application Data
(60) Provisional application No. 62/200,864, filed on Aug. 4, 2015.

(51) Int. Cl. A61K 31/185; A61K 9/00; A61P 25/28

(52) U.S. Cl. CPC A61K 31/185; A61K 9/0053; A61K 31/42; A61K 2300/00; A61P 25/28

(57) ABSTRACT

Described herein is a method for treating a medical condition in a patient, the method comprising: administering to a patient in need (thereof a pharmaceutical composition comprising a therapeutically effective amount of (i) a first therapeutic agent which is acamprosate or a pharmaceutically acceptable salt thereof and (ii) a second therapeutic agent which is D-cycloserine, a salt of D-cycloserine, an ester of D-cycloserine, an alkylated D-cycloserine, or a precursor of D-cycloserine.

### FIELD OF THE INVENTION

The present invention relates to the treatment of various medical conditions using a combination of acamprosate and d-cycloserine.

### BACKGROUND

3-(Acetylamino)propylsulfonic acid, also referred to as N-acetylhomotaurine or acamprosate:

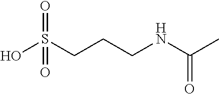

is a derivative of homotaurine, a naturally occurring structural analog of γ-aminobutyric acid (GABA) that appears to affect multiple receptors in the central nervous system (CNS). As an antiglutamatergic agent, acamprosate is believed to exert a neuropharmacological effect as an antagonist of N-methyl-D-aspartate (NMDA) receptors. The mechanism of action is believed to include blocking of the Ca²⁺ channel to slow Ca²⁺ influx and reduce the expression of c-fos, leading to changes in messenger RNA transcription and the concomitant modification to the subunit composition of NMDA receptors in selected brain regions (Zornoza et al., CNS Drug Reviews, 2003, 9 (4), 359-374; and Rammes et al., Neuropharmacology 2001, 40, 749-760). There is also evidence that acamprosate may interact with excitatory glutamatergic neurotransmission in general and as an antagonist of the metabotropic glutamate receptor subtype 5 (mGluR5) in particular (De Witte et al., CNS Drugs 2005, 19 (6), 517-37). The glutamatergic mechanism of action of acamprosate may explain the effects of acamprosate on alcohol dependence and suggests other therapeutic activities such as in neuroprotection.

Acamprosate calcium, marketed as Campral® by Forest Pharma, was first approved by the FDA in 2004. Campral® is indicated for the maintenance of abstinence from alcohol in patients with alcohol dependence who are abstinent at treatment initiation. Treatment with Campral® should be part of a comprehensive management program that includes psychosocial support.

D-Cycloserine (DCS, D-4-amino-3-isoxazolidone):

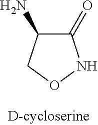

is a derivative of the naturally occurring amino acid D-serine. It is marketed as SEROMYCIN® by Purdue GMP for treatment of active pulmonary and extrapulmonary tuberculosis (including renal disease) when the causative organisms are susceptible to this drug and when treatment with the primary medications (streptomycin, isoniazid, rifampin, and ethambutol) has proved inadequate. Like all antituberculosis drugs, Seromycin should be administered in conjunction with other effective chemotherapy and not as the sole therapeutic agent. Seromycin may be effective in the treatment of acute urinary tract infections caused by susceptible strains of gram-positive and gram-negative bacteria, especially Enterobacter spp. and Escherichia coli.

D-cycloserine is an NMDA receptor partial agonist and acts as co-agonist at the strychnine-insensitive glycine binding site on the NR1 subunit of the NMDA receptor. DCS increases the activation probability of the NMDA receptor; however, it requires the presence of glutamate binding to the receptor in order to exert its effects (Myers, K. M.; Carlezon, W. A. J. “D-cycloserine effects on extinction of conditioned responses to drug-related cues.” Biol. Psychiatry 2012, 71, 947-955). DCS activation enhances NMDA functioning by increasing calcium influx through these receptors without causing neurotoxicity (Olive, M. F.; Cleva, R. M.; Kalivas, P. W.; Malcolm, R. J. “Glutamatergic medications for the treatment of drug and behavioral addictions.” Pharmacol. Biochem. Behav. 2012, 100, 801-810; and Sheinin, A.; Shavit, S.; Benveniste, M. “Subunit specificity and mechanism of action of NMDA partial agonist D-cycloserine,” Neuropharmacology 2001, 41, 151-158). However, DCS is less efficient than the endogenous ligands glycine and D-serine at modulating NMDA receptor function. High doses of DCS displace more efficacious endogenous ligands, and moderate doses of DCS have shown to facilitate NMDA receptor-dependent forms of synaptic plasticity and learning (Myers, K. M.; Carlezon, W. A. J. “D-cycloserine effects on extinction of conditioned responses to drug-related cues.” Biol. Psychiatry 2012, 71, 947-955).

While acamprosate and d-cycloserine have been suggested as monotherapic agents to treat various medical conditions, applicants have discovered that the drugs are useful for combination therapy.

### SUMMARY OF THE INVENTION

The present invention relates to the discovery that the combination of acamprosate and d-cycloserine can be used synergistically to treat various medical conditions.

In a first aspect, the invention provides a method for treating a medical condition in a patient, the method comprising: administering to a patient in need thereof a pharmaceutical composition comprising a therapeutically effective amount of (i) a first therapeutic agent which is acamprosate or a pharmaceutically acceptable salt thereof and (ii) a second therapeutic agent which is D-cycloserine, a salt of D-cycloserine, an ester of D-cycloserine, an alkylated D-cycloserine, or a precursor of D-cycloserine.

In a 1^{st} embodiment, the second therapeutic agent is D-cycloserine.

In a 2^{nd} embodiment, the second therapeutic agent is a salt of D-cycloserine selected from the group consisting of a sodium salt, a potassium salt, a calcium salt, a magnesium salt, a zinc salt, and an ammonium salt of D-cycloserine.

In a 3^{rd} embodiment, the second therapeutic agent is an ester of D-cycloserine having an ester group with 1-20 carbon atoms.

In a 4^{th} embodiment, the second therapeutic agent an alkylated D-cycloserine having an alkyl group with 1-20 carbon atoms.

In a 5^{th} embodiment, the second therapeutic agent is a precursor of D-cycloserine.

In a 6^{th} embodiment, the pharmaceutical composition is administered to the patient for at least one week.

In a 7^{th} embodiment, the pharmaceutical composition is administered to the patient or at least 4 weeks.

In an 8^{th} embodiment, the pharmaceutical composition is administered to the patient for at least 6 weeks.

In a 9^{th} embodiment, the pharmaceutical composition is administered to the patient for at least 8 weeks.

In a 10^{th} embodiment, the pharmaceutical composition is administered to the patient at least once daily.

In an 11^{th} embodiment, the pharmaceutical composition is administered to the patient in two doses per day.

In a 12^{th} embodiment, the pharmaceutical composition is administered to the patient in three doses per day.

In a 13^{th} embodiment, the pharmaceutical composition is administered to the patient in four doses per day.

In a 14^{th} embodiment, the pharmaceutical composition is administered by a route selected from the group consisting of oral, intravenous, trans-mucosal, pulmonary, transdermal, ocular, buccal, sublingual, intraperitoneal, intrathecal, and intramuscular routes.

In a 15^{th} embodiment, the pharmaceutical composition is administered by an oral route.

In a 16^{th} embodiment, the first therapeutic agent is administered in a dose equivalent to 100-2500 mg of acamprosate calcium.

In a 17^{th} embodiment, the second therapeutic agent is administered in a dose equivalent to 105-500 mg of D-cycloserine.

In a 18^{th} embodiment, the second therapeutic agent is administered in a dose equivalent to 125-400 mg of D-cycloserine.

In a 19^{th} embodiment, the second therapeutic agent is administered in a dose equivalent to 150-300 mg of D-cycloserine.

In an 20^{th} embodiment, the medical condition is age-related cognitive impairment, Mild Cognitive Impairment (MCI), dementia, Alzheimer's Disease (AD), prodromal AD, post-traumatic stress disorder (PTSD), schizophrenia, bipolar disorder, amyotrophic lateral sclerosis (ALS), cancer-therapy-related cognitive impairment, mental retardation, Parkinson's disease (PD), autism, compulsive behavior, or substance addiction.

In a 21^{st} embodiment, the medical condition is Alcohol dependence, tinnitus, sleep apnea, Parkinson's disease, levodopa-induced dyskinesias in Parkinson's disease, Alzheimer's disease, Huntington's disease, Amyotrophic lateral sclerosis, Cortical spreading depression, migraine, schizophrenia, Anxiety, tardive dyskinesia, spasticity, multiple sclerosis, various types of pain, or binge eating.

In a 22^{nd} embodiment, the medical condition is an Autism Spectrum Disorders, Pervasive Development Disorder—Not Otherwise Specified, Idiopathic Autism, Fragile X Syndrome, Asperger's Syndrome, Rhett's Syndrome, Childhood disintegrative disorder as further referenced in Diagnostic and Statistical Manual of Mental Disorders IV.

In a 23^{rd} embodiment, the medical condition is Fragile X syndrome.

In a 24^{th} embodiment, the medical condition is an autism spectrum disorder.

In a 25^{th} embodiment, the medical condition is a neurotransmission or cognitive disorder that is characterized as a glutamate-GABA imbalance, a disorder characterized with disrupted or dysregulated ERK signaling pathway, or rasopathies resulting in abnormalities in brain development, learning, memory or cognition.

In a second aspect, the invention provides a method for treating a medical condition in a patient, the method comprising: co-administering to a patient in need thereof (i) a therapeutically effective amount of a first therapeutic agent which is acamprosate or a pharmaceutically acceptable salt thereof and (ii) a therapeutically effective amount of a second therapeutic agent which is D-cycloserine, a salt of D-cycloserine, an ester of D-cycloserine, an alkylated D-cycloserine, or a precursor of D-cycloserine.

In a 1^{st} embodiment, the first and second therapeutic agents are administered in a single dosage form.

In a 2^{nd} embodiment, the single dosage form is suitable for oral administration.

In a 3^{rd} embodiment, the single dosage form is a tablet, capsule, oral suspension, or sprinkle formulation.

In a 4^{th} embodiment, the first and second therapeutic agents are homogeneously dispersed in the single dosage form.

In a 5^{th} embodiment, the first and second therapeutic agents are heterogeneously dispersed in the single dosage form.

In a 6^{th} embodiment, the first and second therapeutic agents are in different layers or sections of the single dosage form.

In a 7^{th} embodiment, the first and second therapeutic agents are co-administered in different dosage forms.

In a 8^{th} embodiment, the first therapeutic agent is administered as a tablet, capsule, oral suspension, or sprinkle formulation, and the second therapeutic agent is co-administered as a separate tablet, capsule, oral suspension or sprinkle formulation.

In a 9^{th} embodiment, the first therapeutic agent is in a first sprinkle formulation and the second therapeutic agent is in a second sprinkle formulations, wherein the first and second sprinkle formulations are in separate sachets.

In a 10^{th} embodiment, the first therapeutic agent is in a first sprinkle formulation and the second therapeutic agent is in a second sprinkle formulation, wherein the first and second sprinkle formulations are combined in a single sachet.

In a third aspect, the present invention provides a pharmaceutical composition comprising: (i) a first therapeutic agent which is acamprosate or a pharmaceutically acceptable salt thereof and (ii) a second therapeutic agent which is D-cycloserine, a salt of D-cycloserine, an ester of D-cycloserine, an alkylated D-cycloserine, or a precursor of D-cycloserine.

It is contemplated that all allowable combinations of the various aspects and embodiments described above and elsewhere within this application are contemplated as further aspects and embodiments of the invention.

### DETAILED DESCRIPTION

AS USED HEREIN:

“Autism” refers to a state of mental introversion characterized by morbid self-absorption, social failure, language delay, and stereotyped behavior. Patients can be diagnosed as suffering from autism by using the DSM-IV criteria.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. When the active ingredient is acamprosate, the preferred salt is the calcium salt.

“Pharmaceutical composition” refers to at least one active ingredient and at least one pharmaceutically acceptable vehicle with which at least one active ingredient is administered to a subject.

“Salt” refers to a chemical compound consisting of an assembly of cations and anions. Salts of a compound of the present disclosure include stoichiometric and non-stoichiometric forms of the salt. In certain embodiments, because of its potential use in medicine, salts of an active ingredient are pharmaceutically acceptable salts.

“Sprinkle formulation” refers to enteric-coated beads or pellets which can be spherical in shape and is currently defined by the FDA to be 0.82 mm to 3.04 mm (+ or −10% variation) in size and can be administered orally with food with or without chewing. Sprinkles can be manufactured in several shapes such as cylindrical, cylindrical with round ends, dumb-bell, ellipsoid or spherical in shape. See “Guidance for Industry Size of Beads in Drug Products Labeled for Sprinkle” U.S. Department of Health and Human Services Food and Drug Administration Center for Drug Evaluation and Research (CDER) May 2012 CMC Rev. 1.

“Sachet” is a small flexible package made by bonding to layers together on all four sides. Often refers to single-use, sealed, flexible aluminum pouches which contains a dose of the formulation of which could be presented as a liquid, powder, cream, paste or granule.

“Subject” and “patient” refer to a mammal, for example, a human.

“Treating” or “treatment” of any disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the subject. In certain embodiments, “treating” or “treatment” refers to delaying the onset of the disease or at least one or more symptoms thereof in a subject which may be exposed to or predisposed to a disease or disorder even though that subject does not yet experience or display symptoms of the disease.

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the subject to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a subject. A therapeutically effective dose may vary from compound to compound, and from subject to subject, and may depend upon factors such as the condition of the subject and the route of delivery. A therapeutically effective dose may be determined in accordance with routine pharmacological procedures known to those skilled in the art.

In all of the methods and compositions of the invention, D-cycloserine (or modified versions thereof) can be substituted with a modified version of the amino acid, such as a salt, ester, alkylated form, or a precursor of the amino acid. For example, the amino acid can be in the form of a sodium salt, potassium salt, calcium salt, magnesium salt, zinc salt, or ammonium salt. Such salt forms D-cycloserine can be made in accordance with conventional methods (see, e.g., Organic Chemistry, pgs. 822-823, Morrison and Boyd, ed., Fifth Edition, Allyn and Bacon, Inc., Newton, Mass.). Other modified forms of D-cycloserine also can be used in the methods and compositions of the invention. For example, the carboxy group of the amino acid can be converted to an ester group by reaction with an alcohol in accordance with standard esterification methods (Id. at 841-843). For example, alcohols having 1-20 carbon atoms can be used to produce an ester of D-cycloserine for use in the invention (e.g., methyl-, ethyl-, propyl-, isopropyl-, butyl-, isobutyl-, sec-butyl-, tert-butyl-, pentyl-, isopentyl-, tert-pentyl-, hexyl-, heptyl-, octyl-, decyl-, dodecyl-, tetradecyl-, hexadecyl-, octadecyl-, and phenyl-alcohols can be used). In another variation, the amino group of the amino acid can be alkylated, using conventional methods, to produce a secondary or tertiary amino group by ammonolysis of halides or reductive amination (Id. at 939-948). For example, an alkyl group having 1-20 carbon atoms can be added to the amino acid to produce an alkylated amino acid (e.g., methyl-, ethyl-, propyl-, isopropyl-, butyl-, isobutyl-, sec-butyl-, tert-butyl-, pentyl-, isopentyl-, tert-pentyl-, hexyl-, heptyl-, decyl-, dodecyl-, tetradecyl-, hexadecyl-, octadecyl- and phenyl-groups can be added to the amino acid).

### Overview

D-cylcoserine (DCS) is the dexatrorotary form of cycloserine, an approved FDA drug with an extensive safety record was used previously for the treatment of tuberculosis. DCS crosses the blood-brain barrier and is a partial agonist at the glycine site of the N-methyl-D-aspartate (NMDA) receptor. NMDA receptors are involved in synaptic plasticity, learning, and memory (see J. Ren et al., “The effects of intra-hippocampal microinfusion of d-cycloserine on fear extinction, and the expression of NMDA receptor subunit NR2B and neurogenesis in the hippocampus in rats”. Progress In Neuro-Psychopharmacology And Biological Psychiatry 44: 257-264 (2013); K. Baker et al., “D-cycloserine does not facilitate fear extinction by reducing conditioned stimulus processing or promoting conditioned inhibition to contextual cues,” Learning & Memory 19 (10): 461-469 (2012)). DCS binds to the strychnine-insensitive glycine binding site on the NR1 NMDA receptor subunit increasing receptor activation to levels of 40-50% maximum stimulation during exposure to glycine alone. (see S. Dravid et al., “Structural determinants of D-cycloserine efficacy at the NR1/NR2C NMDA receptors,” The Journal Of Neuroscience: The Official Journal Of The Society For Neuroscience 30 (7): 2741-2754 (2010); and W. F. Hood et al., “D-cycloserine: a ligand for the N-methyl-D-aspartate coupled glycine receptor has partial agonist characteristics,” Neurosci Lett. March 13; 98 (1):91-5 (1989)). DCS has properties consistent as a partial agonist at the glycine activation site of the NMDA glutamate receptor.

In animal models, DCS administered as a single dose reversed cognitive impairment produced by hippocampal lesions, anticholinergic agents, and early social deprivation. In healthy animals, DCS enhanced extinction of conditioned fear, performance on maze tasks, and visual recognition memory (see D. Quartermain et al., “Acute but not chronic activation of the NMDA-coupled glycine receptor with d-cycloserine facilitates learning and retention,” Eur J Pharm., 257:7-12 (1994); N. Matsuoka and T. G. Aigner, “D-cycloserine, a partial agonist at the glycine site coupled to N-methyl-D-aspartate receptors, improves visual recognition memory in rhesus monkeys,” J Pharmacol Exp Ther., 278:891-897 (1996)).

When DCS is used in conjunction with cognitive behavioral therapy, it helps with fear extinction in an array of anxiety and stress related disorders (see R. A. Nicoll and R. C. Malenka, “Expression mechanisms underlying NMDA receptor-dependent long-term potentiation,” Ann N Y Acad Sci., 1999; 868:515-525 (1999); Y. P. Tang et al., “Genetic enhancement of learning and memory in mice,” Nature, 401:63-69 (1999)). DCS has been shown to not affect performance during training; instead, selectively improving memory for new learning assessed 24 hours after training (8), (see E. Santini et al., “Consolidation of extinction learning involves transfer from NMDA-independent to NMDA-dependent memory,” J. Neurosci., 21:9009-9017 (2001)). Further research suggests DCS enhances memory consolidation but appears to be limited to novel learning (see J. M. Langton and R. Richardson, “D-cycloserine facilitates extinction the first time but not the second time: an examination of the role of NMDA across the course of repeated extinction sessions,” Neuropsychopharmacology, 33:3096-3102 (2008)). It was further reported that tachyphylaxis rapidly develops with repeated dosing of DCS. (see A. S. Parnas et al., “Effects of multiple exposures to D-cycloserine on extinction of conditioned fear in rats,” Neurobiol Learn Mem., 2005; 83:224-231(2005)).

Acamprosate has pleiotropic effects including potential modulation of NMDA glutamate receptor activity While DCS has activity at the neutral amino acid site (commonly known as the glycine receptor site) on the NMDA receptor, acamprosate has been demonstrated to have activity at the polyamine site on the NMDA receptor. The site of acamprosate activity is the same site as where NMDA receptor agonists including NMDA, glutamate, aspartate, and homocysteate bind to the receptor. Acamprosate is postulate to act as a partial agonist on the polyamine NMDA receptor site having net antagonist effects at high polyamine concentrations and agonist effects at low concentrations. This acamprosate effect is similar in concept to the partial agonist effects of DCS, but is at a different and unique binding site of the NMDA receptor complex. Essentially DCS and acamprosate have potentially synergistic or parallel partial agonist capacity, one drug acting at the glycine NMDA site (DCS) and one via the polyamine NMDA site (acamprosate). (see, e.g., M. al Qatari et al., “Mechanism of action of acamprosate. Part II. Ethanol dependence modifies effects of acamprosate on NMDA receptor binding in membranes from rat cerebral cortex,” Alcohol Clinical Experimental Research. 1998 June; 22 (4):810-4; M. al Qatari et al., “Acamprosate is neuroprotective against glutamate-induced excitotoxicity when enhanced by ethanol withdrawal in neocortical cultures of fetal rat brain.” Alcohol Clinical Experimental Research. 2001 September; 25(9):1276-83; K. Mann et al., “Acamprosate: recent findings and future research directions,” Alcohol Clinical Experimental Research, 2008 July; 32 (7):1105-10, doi: 10.1111/j.1530-0277.2008.00690.x; O. Pierrefiche et al., “Biphasic effect of acamprosate on NMDA but not on GABAA receptors in spontaneous rhythmic activity from the isolated neonatal rat respiratory network,” Neuropharmacology. 2004 July; 47 (1):35-45. PMID:15165832; and M. Naassila et al., “Mechanism of action of acamprosate. Part I. Characterization of spermidine-sensitive acamprosate binding site in rat brain,” Alcohol Clinical Experimental Research., 13 1998 June; 22 (4):802-9).

Overall, partial agonism at NMDA receptors provides an opportunity to harness the pro-learning, memory and synaptic plasticity effects of NMDA activity at low levels of endogenous agonist presence while also providing a neuroprotective effect at high endogenous agonist levels. DCS and acamprosate have unique and complementary partial agonist activity on the NMDA receptor complex thus broadening the ability to modulate this receptors activity during potential exposure to glycine or polyamine endogenous agonism.

### Medicinal Uses

In some embodiments, this disclosure is directed to the use of an acamprosate oral pellet formulations in the manufacture of a medicament for use in methods of treatment in any neurotransmission or cognitive disorder that is characterized as a glutamate-GABA imbalance, any disorder characterized with disrupted or dysregulated ERK signaling pathway or rasopathies resulting in abnormalities in brain development, learning, memory and cognition. To include but not limited to, Autism Spectrum Disorders, Pervasive Development Disorders—Not Otherwise Specified, Idiopathic Autism, Fragile X Syndrome, Asperger's Syndrome, Rhett's Syndrome, Childhood Disintegrative Disorder as further referenced in Diagnostic and Statistical Manual of Mental Disorders V, Alcohol dependence, tinnitus, sleep apnea, Parkinson's Disease, levodopa-induced dyskinesias in Parkinson's Disease, Alzheimer's Disease, Huntington's Disease, Amyotrophic Lateral Sclerosis, Cortical spreading depression, migraine, schizophrenia, anxiety, tardive dyskinesia, spasticity, multiple sclerosis, various types of pain, or binge eating, subjects having or at risk for age-related cognitive impairment, Mild Cognitive impairment (MCI), dementia, Alzheimer's Disease (AD), prodromal AD, post traumatic stress disorder (PTSD), bipolar disorder, amyotrophic lateral sclerosis (ALS), cancer-therapy-related cognitive impairment, compulsive behavior, and substance addiction.

Also provided herein is methods of treating a subject with Fragile X syndrome, Autism Spectrum Disorders, Down's syndrome, a neurological disorder and/or mental retardation in order to diminish, halt, ameliorate or prevent one or more of the neurological deficiencies or symptoms associated with the disorder (e.g., benign childhood epilepsy, temporal lobe epilepsy, visual spatial defects, anxiety, aggression, hyperactivity, agitation, repetitive behaviors, abnormal or limited social interactions, language and learning difficulties, and/or combinations thereof). In certain embodiments, children with mental retardation, Autism Spectrum Disorders, Down's Syndrome and Fragile X Syndrome can be treated with a formulation of the invention. The children can be treated during infancy (between about 0 to about 1 year of life), childhood (the period of life between infancy and puberty) and during puberty (between about 8 years of life to about 18 years of life).

In certain embodiments, the methods disclosed herein can be used to treat adults (greater than about 18 years of life) having mental retardation, Fragile X Syndrome, Autism Spectrum Disorders and/or Down's Syndrome. In certain embodiments, anxiety and epilepsy in subjects (both children and adults) having Fragile X Syndrome, Autism Spectrum Disorders, mental retardation and/or Down's syndrome can be treated by administering to the subjects a formulation of the invention.

### Dosing and Administration

In all of the methods of the invention, appropriate dosages of D-cycloserine (or modified versions thereof) and acamprosate can readily be determined by those of ordinary skill in the art of medicine by monitoring the patient for signs of disease amelioration or inhibition, and increasing or decreasing the dosage and/or frequency of treatment as desired.

The amount of an active ingredient that will be effective in the treatment of specific medical condition in a subject will depend, in part, on the nature of the condition and can be determined by standard clinical techniques known in the art. In addition, in vitro or in vivo assays may be employed to help identify optimal dosage ranges. A therapeutically effective amount of an active ingredient to be administered may also depend on, among other factors, the subject being treated, the weight of the subject, the severity of the disease, the manner of administration, and the judgment of the prescribing physician.

In one suitable method of treatment, the pharmaceutical composition is administered to the patient at least once daily for at least one week. If desired, the pharmaceutical composition can be administered to the patient in more than one dose per day (e.g., 2, 3, or 4 doses). Generally, the patient is treated for at least one week; typically, the patient is treated for at least several weeks (e.g., at least 4, 6, or 8 weeks) or months (e.g., at least 4, 8, or 12 months). If necessary, the treatment can continue indefinitely to keep the patient's symptoms under control throughout his or her life.

In the methods of treatment for autism, e.g., pharmaceutical composition containing D-cycloserine in an amount equivalent to a dosage of 105 to 500 mg/day is administered to a patient in need of such treatment. For example, the dosage can be in an amount of 125 to 400 mg, such as 150 to 300 mg (e.g., 175 mg, 200 mg, 225 mg, or 250 mg). D-cycloserine (D-4-amino-3-isoxazolidinone) is commercially available from Eli Lilly, Inc. (Indianapolis, Ind.). Generally, treatment continues for at least one week and can continue for several years or life-long as needed to control the patient's symptoms.

The pharmaceutical compositions can be administered to the patient by any, or a combination, of several routes, such as oral, intravenous, trans-mucosal (e.g., nasal, vaginal, etc.), pulmonary, transdermal, ocular, buccal, sublingual, intraperitoneal, intrathecal, intramuscular, or long term depot preparation.

Oral pharmaceutical formulations, such as tablets, capsule, sprinkle formulations, and oral suspensions, are preferred. Solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate and alginic acid. Tablet binders that may be used include, without limitation, acacia, methylcellulose, sodium carboxymethylcellulose, polyvinylpyrrolidone (Povidone), hydroxypropyl methylcellulose, sucrose, starch, and ethylcellulose.

In some embodiment, embodiment, acamprosate and d-cycloserine are administered as two separate oral formulations, each containing an effective amount of one of the actives. For example, acamprosate can be administered as a tablet, capsule, oral suspension, or sprinkle formulation formulation, with d-cydloserine co-administered as a separate tablet, capsule, oral suspension or sprinkle formulation. Specifically, acamprosate and d-cycloserine can be administered as independent sprinkle formulations in separate sachets. Alternatively, the two sprinkle formulations can be combined in the same cachet for administration at the same time. Likewise, acamprosate can be administered as a sprinkle formulation with d-cycloserine co-administered as a tablet. The term “co-administration” is intended to include taking the two medications at or about the same time or, alternatively, during the same round of therapy.

In other embodiments, acamprosate and d-cycloserine are administered as a single oral formulation containing an effective amount of each active. For example, the combination can be administered as a tablet, capsule, oral suspension, or sprinkle formulation. It will appreciated that the two drugs can be combined in any manner known in the art. The two actives can be homogeneously dispersed in the dosage form, or heterogeneously dispersed therein. For example, the two actives can be in different layers of a bi-layer tablet, of in different sections of a tablet-in-tablet formulation. Similarly, the two actives can be in different layers or sections of the pellets of a capsule or sprinkle formulation.

1. A method for treating a medical condition in a patient, the method comprising: administering to a patient in need thereof a pharmaceutical composition comprising a therapeutically effective amount of (i) a first therapeutic agent which is acamprosate or a pharmaceutically acceptable salt thereof and (ii) a second therapeutic agent which is D-cycloserine, a salt of D-cycloserine, an ester of D-cycloserine, an alkylated D-cycloserine, or a precursor of D-cycloserine, or a combination thereof.

2. The method of claim 1, wherein the second therapeutic agent is D-cycloserine.

3. The method of claim 1, wherein the second therapeutic agent is a salt of D-cycloserine selected from the group consisting of a sodium salt, a potassium salt, a calcium salt, a magnesium salt, a zinc salt, and an ammonium salt of D-cycloserine.

4. The method of claim 1, wherein the second therapeutic agent is an ester of D-cycloserine having an ester group with 1-20 carbon atoms.

5. The method of claim 1, wherein the second therapeutic agent is an alkylated D-cycloserine having an alkyl group with 1-20 carbon atoms.

6. The method of claim 1, wherein the second therapeutic agent is a precursor of D-cycloserine.

7. The method of claim 1, wherein the pharmaceutical composition is administered to the patient for at least one week.

8.-10. (canceled)

11. The method of claim 1, wherein the pharmaceutical composition is administered to the patient at least once daily.

12. The method of claim 11, wherein the pharmaceutical composition is administered to the patient in two, three, or four doses per day.

13.-14. (canceled)

15. The method of claim 1, wherein, the pharmaceutical composition is administered by a route selected from the group consisting of oral, intravenous, trans-mucosal, pulmonary, transdermal, ocular, buccal, sublingual, intraperitoneal, intrathecal, and intramuscular routes.

16. The method of claim 15, wherein the pharmaceutical composition is administered by an oral route.

17. The method of claim 1, wherein the first therapeutic agent is administered in a dose equivalent to 100-2500 mg of acamprosate calcium.

18. The method of claim 17, wherein the second therapeutic agent is administered in a dose equivalent to 105-500 mg of D-cycloserine.

19. The method of claim 18, wherein the second therapeutic agent is administered in a dose equivalent to 125-400 mg of D-cycloserine.

20. The method of claim 19, wherein the second therapeutic agent is administered in a dose equivalent to 150-300 mg of D-cycloserine.

21. The method of claim 1, wherein the medical condition is age-related cognitive impairment, Mild Cognitive Impairment (MCI), dementia, Alzheimer's Disease (AD), prodromal AD, post-traumatic stress disorder (PTSD), schizophrenia, bipolar disorder, amyotrophic lateral sclerosis (ALS), cancer-therapy-related cognitive impairment, mental retardation, Parkinson's disease (PD), autism, compulsive behavior, substance addiction, alcohol dependence, tinnitus, sleep apnea, Parkinson's disease, one or more levodopa-induced dyskinesias in Parkinson's disease, Huntington's disease, cortical spreading depression, migraine, anxiety, tardive dyskinesia, spasticity, multiple sclerosis, pain, binge eating, an autism spectrum disorder, Pervasive Development Disorder—Not Otherwise Specified, Idiopathic Autism, Fragile X Syndrome, Asperger's Syndrome, Rhett's Syndrome, or Heller syndrome.

22.-23. (canceled)

24. The method of claim 23, wherein the medical condition is Fragile X syndrome.

25. The method of claim 23, wherein the medical condition is an autism spectrum disorder.

26. The method of claim 1, wherein the medical condition is a neurotransmission or cognitive disorder that is characterized as a glutamate-GABA imbalance, a disorder characterized with disrupted or dysregulated ERK signaling pathway, or rasopathies resulting in abnormalities in brain development, learning, memory or cognition.

27. A pharmaceutical composition comprising: (i) a first therapeutic agent which is acamprosate or a pharmaceutically acceptable salt thereof and (ii) a second therapeutic agent which is D-cycloserine, a salt of D-cycloserine, an ester of D-cycloserine, an alkylated D-cycloserine, or a precursor of D-cycloserine, or a combination thereof.
